(19) United States
(12) Patent Application Publication
(10) Pub. No.: US 2015/0374689 A1
(43) Pub. Date: Dec. 31, 2015

(54) Use of Levosimendan to Treat Left Ventricular Systolic Dysfunction in Patients Undergoing Cardiac Surgery Requiring Cardiopulmonary Bypass

(71) Applicant: TENAX Therapeutics, Inc., Morrisville, NC (US)

(72) Inventors: John Kelley, Chester, NJ (US); Doug Randall, Belle Mead, NJ (US); Douglas Hay, Ottsville, PA (US)

(73) Assignee: TENAX Therapeutics, Inc., Morrisville, NC (US)

(21) Appl. No.: 14/751,476

(22) Filed: Jun. 26, 2015

Related U.S. Application Data
(60) Provisional application No. 62/019,328, filed on Jun. 30, 2014.
(60) Provisional application No. 62/024,345, filed on Jul. 14, 2014.

(51) Int. Cl. A61K 31/50

(52) U.S. Cl. CPC A61K 31/50

(57) ABSTRACT

Methods of (i) reducing morbidity and/or mortality in a human patient undergoing cardiac surgery; (ii) preventing, or reducing the risk of development of, LCOS in a human patient undergoing cardiac surgery; or (iii) reducing the risk of, intensity of, or occurrence of, one or more postoperative adverse events in a human patient undergoing cardiac surgery. The methods can involve (a) a first period of administering to the patient levosimendan for about 1 hour, in which the administration of levosimendan during the first period is initiated: (i) before skin incision for the cardiac surgery, and (ii) at an infusion dose of about 0.2 μg/kg/min; and (b) a second period of administering to the patient levosimendan for about 23 hours, in which the administration of levosimendan during the second period is initiated at an infusion dose of about 0.1 μg/kg/min.

### CROSS REFERENCE TO RELATED APPLICATIONS

This application claims the benefit of U.S. provisional application Ser. No. 62/019,328, filed on Jun. 30, 2014, and U.S. provisional application Ser. No. 62/024,345, filed on Jul. 14, 2014. Both provisional applications are hereby incorporated by reference.

### FIELD OF THE INVENTION

The present invention is in the field of treatment of human patients undergoing cardiac surgery. In particular, the present invention relates to (i) reducing morbidity and/or mortality in a human patient undergoing cardiac surgery; (ii) preventing, or reducing the risk of development of, LCOS in a human patient undergoing cardiac surgery; or (iii) reducing the risk of, intensity of, or occurrence of, one or more postoperative adverse events in a human patient undergoing cardiac surgery.

### BACKGROUND OF THE INVENTION

Over the past several decades, the risk profile of patients undergoing cardiac surgery has increased in part due to mounting evidence suggesting that patients with coronary artery disease and reduced left ventricular function benefit from surgical revascularization over medical therapy. (O'Conner 2002; Velazquez 2011). Patients with impaired left ventricular function undergoing cardiac surgery have relatively high rates of mortality and other adverse events, including need for dialysis or mechanical support. Shahian et al. used the Society of Thoracic Surgeons National Adult Cardiac Surgery Database (STS NCD) to derive a risk model for cardiac surgery using the 700,000 isolated coronary artery bypass graft (CABG) procedures performed between 2006 and 2008. They found that each 10-unit decrease in left ventricular ejection fraction (LVEF) was associated with a 19% increase in the odds of death, and an 8% increase in the odds of developing renal failure or new dialysis. (Shahian et al. 2009).

Medical practice associated with cardiac surgery has evolved in the last decade to aggressively manage patients in order to minimize the manifestations of, in particular, low cardiac output syndrome (LCOS) in the peri- and post-surgical periods. Yet, despite advances, LCOS remains a substantial risk in cardiac surgery. (Masse et al. 2005). LCOS is the often transient and reversible condition of reduced cardiac output which leads to a decrease in systemic oxygen delivery. (Kumar et al. 2010). It may be characterized as the need for postoperative pharmacologic stimulation of ventricular contraction and/or mechanical circulatory support to maintain systolic blood pressure (SBP) over 90 mmHg or a cardiac index above 2.2 L/min/m². (Góngora et al. 2008). The effects of LCOS include multi-organ dysfunction, an increase in patient morbidity, prolonged hospitalization and possible mortality. (Kumar et al. 2010). It often follows the use of cardiopulmonary bypass (CPB) during cardiac surgery (Kumar et al. 2010) and has been described in 3-14% of CABG cases, and is associated with a 10-15 fold increase in mortality (Algarni 2011).

Transient myocardial depression typically associated with cardiac surgery using CPB places patients at risk of LCOS. The risk for LCOS is increased in patients with pre-existing low LVEF, diabetes, prior CABG surgery, emergency surgery, as well as for those patients who are female and/or older than 70 years of age. These higher risk patient groups represent a fast growing subset of the cardiac surgery patient population.

Currently, there are no pharmacologic therapies approved to reduce the incidence of LCOS or the associated mortality and/or morbidity. When cardiac function declines, pharmacologic and mechanical assist therapies are employed to optimize hemodynamic performance to ensure CPB weaning success and minimize peripheral tissue/organ damage. This management is particularly important in those cardiac surgery patients whose hearts have little margin for further diminished capacity, i.e., the cardiac surgery population at high risk for LCOS characterized by low LVEF.

### SUMMARY OF THE INVENTION

In one aspect, the present invention relates to a method of reducing morbidity and/or mortality in a human patient undergoing cardiac surgery, the method comprising (a) a first period of administering to the human patient levosimendan for about 1 hour, in which the administration of levosimendan during the first period is initiated (i) before skin incision for the cardiac surgery, and (ii) at an infusion dose of about 0.2 μg/kg/min; and (b) a second period of administering to the human patient levosimendan for about 23 hours, in which the administration of levosimendan during the second period is initiated at an infusion dose of about 0.1 μg/kg/min.

In another aspect, the present invention relates to a method of preventing, or reducing the risk of development of, LCOS in a human patient undergoing cardiac surgery, comprising (a) a first period of administering to the human patient levosimendan for about 1 hour, in which the administration of levosimendan during the first period is initiated (i) before skin incision for the cardiac surgery, and (ii) at an infusion dose of about 0.2 μg/kg/min; and (b) a second period of administering to the human patient levosimendan for about 23 hours, in which the administration of levosimendan during the second period is initiated at an infusion dose of about 0.1 μg/kg/min.

In another aspect, the present invention relates to a method of reducing the risk of, intensity of, or occurrence of, one or more postoperative adverse events in a human patient undergoing cardiac surgery, the method comprising (a) a first period of administering to the human patient levosimendan for about 1 hour, in which the administration of levosimendan during the first period is initiated (i) before skin incision for the cardiac surgery, and (ii) at an infusion dose of about 0.2 μg/kg/min; and (b) a second period of administering to the human patient levosimendan for about 23 hours, in which the administration of levosimendan during the second period is initiated at an infusion dose of about 0.1 μg/kg/min.

In another aspect, the present invention relates to a method of reducing morbidity and/or mortality in a human patient undergoing cardiac surgery, the method comprising (a) a first period of administering to the human patient levosimendan for about 1 hour, in which the administration of levosimendan during the first period is initiated (i) before skin incision for the cardiac surgery, and (ii) at an infusion dose of about 0.2 μg/kg/min, and in which the about 0.2 μg/kg/min infusion dose is decreased to one or more lower doses; and (b) a second period of administering to the human patient levosimendan for about 23 hours, in which the administration of levosimendan during the second period is initiated at an infusion dose selected from the one or more lower doses administered during the first period.

In another aspect, the present invention relates to a method of preventing, or reducing the risk of development of, LCOS in a human patient undergoing cardiac surgery, comprising (a) a first period of administering to the human patient levosimendan for about 1 hour, in which the administration of levosimendan during the first period is initiated (i) before skin incision for the cardiac surgery, and (ii) at an infusion dose of about 0.2 μg/kg/min, and in which the about 0.2 μg/kg/min infusion dose is decreased to one or more lower doses; and (b) a second period of administering to the human patient levosimendan for about 23 hours, in which the administration of levosimendan during the second period is initiated at an infusion dose selected from the one or more lower doses administered during the first period.

Another aspect of the present invention relates to a method of reducing the risk of, intensity of, or occurrence of, one or more postoperative adverse events in a human patient undergoing cardiac surgery, the method comprising (a) a first period of administering to the human patient levosimendan for about 1 hour, in which the administration of levosimendan during the first period is initiated (i) before skin incision for the cardiac surgery, and (ii) at an infusion dose of about 0.2 μg/kg/min, and in which the about 0.2 μg/kg/min infusion dose is decreased to one or more lower doses; and (b) a second period of administering to the human patient levosimendan for about 23 hours, in which the administration of levosimendan during the second period is initiated at an infusion dose selected from the one or more lower doses administered during the first period.

An aspect of the present invention relates to the use of levosimendan to (i) reduce morbidity and/or mortality in a human patient undergoing cardiac surgery; (ii) prevent, or reduce the risk of development of, LCOS in a human patient undergoing cardiac surgery; or (iii) reduce the risk of, intensity of, or occurrence of, one or more postoperative adverse events in a human patient undergoing cardiac surgery.

Yet another aspect of the present invention relates to a use of an amount of levosimendan in the preparation of a medicament for (i) reducing morbidity and/or mortality in a human patient undergoing cardiac surgery; (ii) preventing, or reducing the risk of development of, LCOS in a human patient undergoing cardiac surgery; or (iii) reducing the risk of, intensity of, or occurrence of, one or more postoperative adverse events in a human patient undergoing cardiac surgery.

Another aspect of the present invention relates to a package or kit, comprising (a) a pharmaceutical composition comprising an amount of levosimendan, and (b) instructions for use of the pharmaceutical composition to (i) reduce morbidity and/or mortality in a human patient undergoing cardiac surgery; (ii) prevent, or reduce the risk of development of, LCOS in a human patient undergoing cardiac surgery; or (iii) reduce the risk of, intensity of, or occurrence of, one or more postoperative adverse events in a human patient undergoing cardiac surgery.

Another aspect of the present invention relates to a pharmaceutical composition comprising levosimendan for use in (i) reducing morbidity and/or mortality in a human patient undergoing cardiac surgery; (ii) preventing, or reducing the risk of development of, LCOS in a human patient undergoing cardiac surgery; or (iii) reducing the risk of, intensity of, or occurrence of, one or more postoperative adverse events in a human patient undergoing cardiac surgery.

### BRIEF DESCRIPTION OF THE FIGURES

For a more complete understanding of the invention, reference is made to the following description and accompanying drawing, as follows:

FIG. 1 is a schematic of the study described in Example 1.

### DETAILED DESCRIPTION OF THE INVENTION

The present invention generally relates to the use of levosimendan in patients undergoing cardiac surgery.

Levosimendan (Simdax®) is a calcium sensitizer with vasodilatory and cardioprotective properties. (Toller 2006). It is potent in the treatment of heart failure and has significant calcium dependent binding to troponin. It is the (−)-enantiomer of [[4-(1,4,5,6-tetrahydro-4-methyl-6-oxo-3-pyridazinyl)phenyl]hydrazono]prop anedinitrile, and methods for its preparation are described in WO 92/12135 and WO 97/35841, which are incorporated herein by reference. Levosimendan is represented by formula (I):

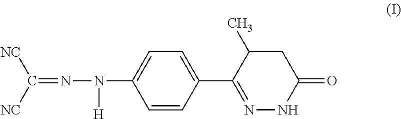 [SMILES: CC1CC(=O)NN=C1c1ccc(NN=C(C#N)C#N)cc1]

Levosimendan is extensively metabolized before excretion into urine and feces. The main pathway is conjugation with glutathione to form inactive metabolites. The minor pathway (approximately 6% of the total levosimendan dose) is reduction in the intestine to an intermediate metabolite OR-1855, which is further acetylated to an active metabolite, OR-1896. (Antila 1999). In patients undergoing cardiac surgery, the formation of the metabolites OR-1855 and OR-1896 has been shown to be delayed. (Kivikko et al. 2002; Eriksson et al. 2009).

The terminal elimination half-life of levosimendan is about 1 hour. The elimination half-life of the metabolite OR-1896 is 70-80 hours in heart failure patients and the maximum concentrations are only seen 2-4 days after starting a 24-hour infusion. (Antila 2007).

Levosimendan has a unique pharmacodynamic profile, which differentiates it from other agents typically used in acute decompensated cardiac patients. The drug increases cardiac contractility through calcium sensitization of troponin C. Unlike other positive inotropes, levosimendan is not associated with substantial increases in oxygen demand. It has been demonstrated as a vasodilator of the arterial and venous circulation through its activity on K_{ATP} channels. In contrast to other inotropes such as milrinone, however, levosimendan is also a vasodilator of the coronary circulation. Further, levosimendan uniquely opens K_{ATP} of mitochondria within cardiomyocytes, an activity associated with reduced apoptosis in nonclinical models and reduced circulating troponin levels in acute decompensated heart failure and cardiac surgery patients.

### Methods/Uses of the Invention

The present invention relates to a method of (i) reducing morbidity and/or mortality in a human patient undergoing cardiac surgery; (ii) preventing, or reducing the risk of development of, LCOS in a human patient undergoing cardiac surgery; or (iii) reducing the risk of, intensity of, or occurrence of, one or more postoperative adverse events in a human patient undergoing cardiac surgery; in which the method comprises (a) a first period of administering to the human patient levosimendan for about 1 hour, in which the administration of levosimendan during the first period is initiated (i) before skin incision for the cardiac surgery, and (ii) at an infusion dose of about 0.2 μg/kg/min; and (b) a second period of administering to the human patient levosimendan for about 23 hours, in which the administration of levosimendan during the second period is initiated at an infusion dose of about 0.1 μg/kg/min.

The about 0.2 μg/kg/min infusion dose initiated during the first period may be decreased to one or more lower infusion doses. For example, the about 0.2 μg/kg/min infusion dose may be decreased to about 0.1 μg/kg/min, or it may be further decreased to about 0.05 μg/kg/min.

The about 0.1 μg/kg/min infusion dose initiated during the second period may be decreased to one or more lower infusion doses. For instance, the about 0.1 μg/kg/min infusion dose may be decreased to about 0.05 μg/kg/min.

The decrease in the infusion dose may be as deemed appropriate, such as in consideration of the best interest of the human patient in accordance with the judgment of a medical practitioner.

The present invention also relates to a method of (i) reducing morbidity and/or mortality in a human patient undergoing cardiac surgery; (ii) preventing, or reducing the risk of development of, LCOS in a human patient undergoing cardiac surgery; or (iii) reducing the risk of, intensity of, or occurrence of, one or more postoperative adverse events in a human patient undergoing cardiac surgery; in which the method comprises (a) a first period of administering to the human patient levosimendan for about 1 hour, in which the administration of levosimendan during the first period is initiated (i) before skin incision for the cardiac surgery, and (ii) at an infusion dose of about 0.2 μg/kg/min, and in which the about 0.2 μg/kg/min infusion dose is decreased to one or more lower doses; and (b) a second period of administering to the human patient levosimendan for about 23 hours, in which the administration of levosimendan during the second period is initiated at an infusion dose selected from the one or more lower doses administering during the first period.

The about 0.2 μg/kg/min infusion dose administered during the first period may be decreased to, for example about 0.1 μg/kg/min, or it may be further decreased to about 0.05 μg/kg/min. The infusion dose initiated during the second period may be at the lowest infusion dose administered during the first period. For instance, if the infusion dose administered during the first period is decreased first to about 0.1 μg/kg/min, and then to about 0.05 μg/kg/min, then the infusion dose initiated during the second period may be about 0.05 μg/kg/min. Alternatively, the infusion dose initiated during the second period may not be at the lowest infusion dose that was administered during the first period.

The decrease in the infusion dose may be as deemed appropriate, such as in consideration of the best interest of the human patient in accordance with the judgment of a medical practitioner.

The present invention relates to the use of levosimendan to (i) reduce morbidity and/or mortality in a human patient undergoing cardiac surgery; (ii) prevent, or reduce the risk of development of, LCOS in a human patient undergoing cardiac surgery; or (iii) reduce the risk of, intensity of, or occurrence of, one or more postoperative adverse events in a human patient undergoing cardiac surgery. The present invention also relates to a use of an amount of levosimendan in the preparation of a medicament for (i) reducing morbidity and/or mortality in a human patient undergoing cardiac surgery; (ii) preventing, or reducing the risk of development of, LCOS in a human patient undergoing cardiac surgery; or (iii) reducing the risk of, intensity of, or occurrence of, one or more postoperative adverse events in a human patient undergoing cardiac surgery. Such reduction of morbidity and/or mortality in a human patient undergoing cardiac surgery, or prevention or reduction of the risk of development of LCOS in a human patient undergoing cardiac surgery, or reduction of the risk of, intensity of, or occurrence of, one or more postoperative adverse events in a human patient undergoing cardiac surgery, are in accordance to the methods of the present invention.

LCOS may be characterized as having a cardiac index of about 2.2 L/min/m² or less, or about 2.0 L/min/m² or less. In certain embodiments, this cardiac index may be at about 2.2 L/min/m² or less, or about 2.0 L/min/m² or less, for about 30 minutes or greater, and despite optimal fluid balance and/or inotropic support (e.g., dobutamine, milrinone, epinephrine, norepinephrine, dopamine, etc.). LCOS may be characterized as having an SBP of about 90 mmHg or less. LCOS may also be identified by the need for either inotropic or mechanical support in order to be weaned from CPB or in the intensive care unit (ICU) because of hemodynamic compromise. Or LCOS may by identified by the need for postoperative pharmacologic stimulation of ventricular contraction, or for mechanical circulatory support, or for both, to maintain SBP greater than about 90 mmHg or a cardiac index greater than about 2.2 L/min/m².

The cardiac surgery that patients are undergoing may be, for example, CABG surgery, CABG with aortic valve surgery, CABG with mitral valve surgery, or isolated mitral valve surgery. Cardiac surgery may also include CABG with aortic valve and mitral valve surgery, tricuspid valve surgery, or mitral valve and tricuspid valve surgery. In certain embodiments, the patients are undergoing a CBP.

In some embodiments, the reduction of morbidity and/or mortality in a human patient undergoing cardiac surgery, or the prevention or reduction of the risk of development of LCOS in a human patient undergoing cardiac surgery, or the reduction of the risk of, intensity of, or occurrence of, a postoperative adverse event in a human patient undergoing cardiac surgery, is after the cardiac surgery.

The one or more postoperative adverse events may be all-cause mortality. For example, the all-cause mortality may be all-cause mortality occurring on or within about 30 days of the cardiac surgery. All-cause mortality through about 30 days covers the peri- and postsurgical period in which cardiac surgery patients are at highest risk.

The one or more postoperative adverse events may also be a myocardial infarction (MI). The MI may be perioperative MI, such as MI occurring on or within about 5 days of the cardiac surgery. MI may be defined by various measurements, as would be understood by a person of ordinary skill in the art. For example, MI may be defined by a blood sample measurement of the biomarker creatine kinase-MB (CK-MB), such as a CK-MB fraction greater than ten times the upper limit of normal (ULN) or greater than about 100 ng/mL, irrespective of electrocardiogram (ECG) changes; or a CK-MB fraction greater than five times the ULN or greater than about 50 ng/mL, along with either new Q waves longer than about 30 ms or left bundle branch block on ECG.

The one or more postoperative adverse events may be a need for dialysis, such as a need that occurs on or within about 30 days of the cardiac surgery. The need for dialysis may result from kidney injury from cardiac surgery and/or LCOS.

In addition, the one or more postoperative adverse events may be use of a mechanical assist device. The use of a mechanical assist device may occur from the start of cardiac surgery to on or within about 5 days of the cardiac surgery, and may occur despite inotropic support (e.g., the use of two inotropes, such as dobutamine, milrinone, epinephrine, norepinephrine, and/or dopamine) and optimal fluid balance. The mechanical assist devices may be employed where the patient's cardiac index persists at less than about 2.2 L/min/m², or at less than about 2.0 L/min/m². For example, such patients may have reduced ejection fraction undergoing cardiac surgery on CPB, or may have post-cardiotomy LCOS, which is generally defined as a patient's inability to maintain a cardiac index at about 2.2 L/min/m² or above, or to maintain a cardiac index at about 2.0 L/min/m² or above. The device may comprise an intra-aortic balloon pump (IABP), left ventricular assist device (LVAD), or extracorporeal membrane oxygenation (ECMO).

In certain embodiments, the one or more postoperative adverse events may be a composite of all-cause mortality or use of a mechanical assist device. In some embodiments, the one or more postoperative adverse events may be a composite of all-cause mortality, MI, need for dialysis, or use of a mechanical assist device.

Further, one or more postoperative adverse events may be postoperative use of one or more secondary inotropes associated with index surgical procedure. The one or more secondary inotropes may include, but are not limited to, dobutamine, milrinone, epinephrine, norepinephrine, dopamine, or a combination thereof.

The one or more postoperative adverse events may be LCOS, such as LCOS defined as a cardiac index at about 2.2 L/min/m² or less or at about 2.0 L/min/m² or less, for about 30 minutes or greater, despite optimal fluid balance and inotropic support.

The one or more postoperative adverse events may also be an increased duration of ICU length of stay (LOS) or increased duration of critical or coronary care unit (CCU) LOS during the index hospitalization.

Administration

Levosimendan may be administered parenterally, such as intravenously.

The administration of levosimendan initiated at the about 0.2 μg/kg/min infusion dose during the first period may be initiated after arterial line insertion, but before skin incision. In certain embodiments, the administration of levosimendan initiated at the about 0.2 μg/kg/min infusion dose during the first period may be initiated no later than the onset of anesthesia. If insertion of a Swan-Ganz catheter is involved in the cardiac surgery, the catheter may be inserted prior to the administration of levosimendan, or as soon as possible after administration of levosimendan is initiated.

In some embodiments, administration of levosimendan in the first period is no earlier than about 24 hours before the cardiac surgery. In certain embodiments, administration of levosimendan in the first period is no earlier than about 18 hours before the cardiac surgery. Administration of levosimendan in the first period may also be no earlier than about 15 hours before the cardiac surgery, or about 12 hours before the cardiac surgery, or about 8 hours before the cardiac surgery, or about 6 hours before the cardiac surgery, or about 5 hours before the cardiac surgery, or about 4 hours before the cardiac surgery, or about 3 hours before the cardiac surgery, or about 2 hours before the cardiac surgery, or about 1 hour before the cardiac surgery.

Levosimendan may be in the form of a diluted infusion solution, such as a diluted infusion solution comprising levosimendan of about 50 μg/mL in 5% dextrose. Levosimendan may also be diluted in normal saline.

The infusion rate may be dependent on the weight of the human patient. For example, the infusion rate may be in accordance to Table 1.

TABLE 1
Infusion rates for the different periods of administration
of a 50 μg/mL preparation of levosimendan infusion
according to certain embodiments of the invention.

| Patients | Infusion rate during | Infusion rate during |
|---|---|---|
| weight | first 60 min | 1 h-24 h |
| (kg) | (mL/h) | (mL/h) |
| 40-59 | 10-13 | 5-7 |
| 60-79 | 14-18 | 7-9 |
| 80-99 | 19-23 | 10-11 |
| 100-114 | 24-26 | 12-13 |
| 115-119 | 28 | 14 |

As another example, the infusion rate may be in accordance to the following Table 2.

TABLE 2
Infusion rates for the different periods of administration
of a 50 μg/mL preparation of levosimendan infusion
according to certain embodiments of the invention.

| | | | Reduced |
|---|---|---|---|
| Patients | Infusion rate during | Infusion rate during | infusion rate as |
| weight | first 60 min (mL/h) at | 1 h-24 h (mL/h) at | needed (mL/h) at |
| (kg) | 0.2 μg/kg/min | 0.1 μg/kg/min | 0.05 μg/kg/min |
| 40-44 | 10 | 5 | 2 |
| 45-49 | 11 | 5 | 3 |
| 50-54 | 12 | 6 | 3 |
| 55-59 | 13 | 7 | 3 |
| 60-64 | 14 | 7 | 4 |
| 65-69 | 16 | 8 | 4 |
| 70-74 | 17 | 8 | 4 |
| 75-79 | 18 | 9 | 5 |
| 80-84 | 19 | 10 | 5 |
| 85-89 | 20 | 10 | 5 |
| 90-94 | 22 | 11 | 5 |
| 95-99 | 23 | 11 | 6 |
| 100-104 | 24 | 12 | 6 |
| 105-109 | 25 | 13 | 6 |
| 110-114 | 26 | 13 | 7 |
| 115-119 | 28 | 14 | 7 |
| 120-124 | 29 | 14 | 7 |
| 125-129 | 30 | 15 | 8 |
| 130-134 | 31 | 16 | 8 |
| 135-139 | 32 | 16 | 8 |
| 140-144 | 34 | 17 | 8 |
| 145-149 | 35 | 17 | 9 |
| 150-154 | 36 | 18 | 9 |
| 155-159 | 37 | 19 | 9 |
| 160-164 | 38 | 19 | 10 |
| 165-169 | 40 | 20 | 10 |

In certain embodiments, the infusion rate for a human patient may be calculated using the following formula (II):

$$infusion rate [ mL \text{/} h ] = \frac{( \begin{matrix}dosage \\ [ mg \text{/} kg \text{/} min ]\end{matrix} ) \times ( \begin{matrix}human patient weight \\ [ kg ]\end{matrix} )}{( \begin{matrix}preparation concentration \\ [ µg \text{/} mL ]\end{matrix} )} \times ( \begin{matrix}duration of infusion \\ [ min ]\end{matrix} ) \qquad ( II )$$

Patient

The human patient may be female or male, and may be 18 years of age or older. In certain embodiments, the human patient may be 70 years of age or older.

The human patient may suffer from one or more other medical conditions. For instance, the human patient may suffer from diabetes, obesity, and/or heart failure.

The human patient may have a pre-existing low LVEF. For instance, the human patient may have a documented LVEF of about 35% or less, or a documented LVEF of about 25% or less. The LVEF may be measured within about 60 days of surgery, and it may be measured by techniques known in the art, such as by ventriculogram, echocardiogram or nuclear scan.

The human patient may have undergone prior surgery. For example, the human patient may have underwent prior cardiac surgery, such as CABG, CABG with aortic valve surgery, CABG with mitral valve surgery, or isolated mitral valve surgery. The human patient may have underwent prior cardiac surgery involving placement on CPB. The human patient may also have underwent prior emergency surgery.

The human patient may be at risk of developing LCOS. For example, the patient may have pre-existing low LVEF as described above. In certain embodiments, the human patient may have a documented LVEF of about 25% or less, measured within about 60 days of the cardiac surgery, and the cardiac surgery is CABG. In some embodiments, the human patient may have a documented LVEF of about 35% or less, measured within about 60 days of the cardiac surgery, and the cardiac surgery is CABG with aortic valve surgery, CABG with mitral valve surgery, or isolated mitral valve surgery. Alternatively, or in addition, the patient may have diabetes, prior CABG surgery, emergency surgery, is female, is older than 70 years of age, is obese, or a combination thereof.

The human patient may be receiving standard of care therapy before surgery, during surgery, after surgery, or a combination thereof. Such standard of care therapy may include, but are not limited to, diuretics, digitalis, angiotensin-converting-enzyme (ACE) inhibitors, nitrates, beta-blockers, antibiotics, analgesics, inotropes, vasopressors, vasopressin, antiarrhythmics, nitrates, nitric oxide, and a combination thereof.

The human patient may have been administered levosimendan previously, or may be naïve to levosimendan.

The human patient may not require preplanned mechanical assist device to be used during or after surgery.

In certain embodiments, the human patient may be placed on CPB during the cardiac surgery.

### Package/Kit and Pharmaceutical Compositions

The present invention also relates to a package or kit, comprising (a) a pharmaceutical composition comprising an amount of levosimendan; and (b) instructions for use of the pharmaceutical composition. The use may be to (i) reduce morbidity and/or mortality in a human patient undergoing cardiac surgery; (ii) prevent, or reduce the risk of development of, LCOS in a human patient undergoing cardiac surgery; or (iii) reduce the risk of, intensity of, or occurrence of, a postoperative adverse event in a human patient undergoing cardiac surgery. Such reduction of morbidity and/or mortality in a human patient undergoing cardiac surgery, or prevention or reduction of the risk of development of LCOS in a human patient undergoing cardiac surgery, or the reduction of the risk of, intensity of, or occurrence of, a postoperative adverse event in a human patient undergoing cardiac surgery, are in accordance to the methods of the present invention.

The present invention further relates to a pharmaceutical composition comprising levosimendan for use in (i) reducing morbidity and/or mortality in a human patient undergoing cardiac surgery; (ii) preventing, reducing the risk of development of, LCOS in a human patient undergoing cardiac surgery; or (iii) reducing the risk of, intensity of, or occurrence of, a postoperative adverse event in a human patient undergoing cardiac surgery. This reduction of morbidity and/or mortality in a human patient undergoing cardiac surgery, or prevention or reduction of the risk of development of LCOS in a human patient undergoing cardiac surgery, or the reduction of the risk of, intensity of, or occurrence of, a postoperative adverse event in a human patient undergoing cardiac surgery, are in accordance to the methods of the present invention.

The pharmaceutical composition may comprise levosimendan and one or more pharmaceutical acceptable excipients. For example, the pharmaceutical composition may be as described in U.S. Pat. Nos. 6,790,673 or 6,943,164, which are incorporated herein by reference. In certain embodiments, the pharmaceutical composition comprises levosimendan, povidone, citric acid, and ethanol. Levosimendan may be present in the pharmaceutical composition in a concentration of about 0.1 mg/mL to about 5 m/mL, or about 2.5 mg/mL.

This invention will be better understood by reference to the Example that follows, but those skilled in the art will readily appreciate that the specific experiment detailed is only illustrative of the invention as described more fully in the claims which follow thereafter.

### EXAMPLES

### Example 1

### Clinical Trial—A Double-Blind, Randomized, Placebo-Controlled Study of Levosimendan in Patients with Left Ventricular Systolic Dysfunction Undergoing Cardiac Surgery Requiring Cardiopulmonary Bypass

### Study Design

A randomized, double-blind, placebo-controlled, multicenter study of levosimendan is conducted in subjects with pre-existing left ventricular systolic dysfunction (documented (i) LVEF≦25% in CABG surgery patients, or (ii) LVEF≦35% in CABG with aortic valve surgery patients, CABG with mitral valve surgery patients, or isolated mitral valve surgery patients) with or without heart failure (NYHA functional Class I-IV), undergoing (1) CABG surgery, (2) CABG with aortic valve surgery, (3) CABG with mitral valve surgery, or (4) isolated mitral valve surgery.

All patients are randomized with planned CPB. Approximately 760 subjects are enrolled in the study. Subjects are randomly assigned to receive either levosimendan or a matching placebo in a 1:1 ratio. The study is divided into a screening phase, a double-blind treatment phase, a double-blind post-treatment and follow-up phase from hospital discharge through Day 30. Day 0 is the day of study drug (levosimendan or matching placebo) initiation. All randomly assigned subjects are followed for 90 days, including subjects who were randomly assigned to treatment but not treated.

In the screening phase, potential subjects are screened to determine eligibility for participation in the study. Before any study-related procedures are performed, the risks of the study are explained to the potential subject, and the subject or his/her legal representative is required to sign an informed consent form. Within 60 days before surgery the subject must have a documented LVEF≦25% (if undergoing CABG surgery), or LVEF≦35% (if undergoing CABG with aortic valve surgery, CABG with mitral valve surgery, or isolated mitral valve surgery). If more than one documented LVEF is available, the one closest to the surgery date is used for screening purposes. At the screening visit and within 24 hours before surgery, an ECG and blood samples for CK and CK-MB and troponins, as well as other routine laboratory tests, are obtained. The blood sample required for determination of baseline creatine kinase (CK), CK-MB fractions and troponins are obtained within 8 hours prior to surgery. All subjects are asked at baseline to provide contact details and additional information when permitted by local regulations, such as name, address, phone numbers, employer (name, address, phone numbers), e-mail address, social security number or equivalent, health insurance provider, health insurance policy number, relatives (name, relationship, address, phone numbers), other contacts who have personal knowledge of the subject (e.g., friends or neighbors), primary care physician, and/or other health care provider (cardiologist, dentist, etc.), in order to ensure Day 30 and Day 90 follow-up. A reliable contact must be made at Day 30 and Day 90 preferably by speaking directly to the subject or to someone who has knowledge of the subject's vital status or by a documented source containing all of the required study relevant information (e.g., dialysis center records, laboratory reports, medical visit records). Following acquisition of baseline information and vital signs measurements, subjects are randomly assigned to a treatment group using the clinical data management system (CDMS), after they have met all of the inclusion criteria and none of the exclusion criteria.

In the double-blind treatment phase, every attempt is made to start treatment with study drug as soon as possible prior to surgery. However, in all cases study drug is initiated after arterial line insertion and before skin incision. If a Swan-Ganz catheter is planned, it is preferable to insert it prior to study drug initiation. Study drug is administered as described below. Subjects who were randomly assigned to treatment but not treated will be followed (a) through Day 5 for perioperative MI and the need for IABP/LVAD/ECMO, (b) through postoperative Day 0 for postoperative MI (Day 6-10) and postoperative dialysis, and (c) the occurrence of all-cause mortality through Day 90. All subjects may receive additional standard of care medications including inotropes, vasopressors, vasopressin, antiarrhythmics, diuretics, nitrates, and/or nitric oxide as needed. Due to the potential hypotensive effects of the study drug, the concomitant use of vasodilatory drugs should be used with caution.

In the post-treatment period through hospital discharge, blood samples are collected for hematology and serum chemistry analysis, vital signs are measured, urine output recorded and health care resource utilization data are recorded. In particular, cardiac biomarkers (CK and CK-MB fractions and cardiac troponin) are obtained at baseline and then q 12 hours for the first 48 hours and then if clinically indicated for ischemic symptoms. Electrocardiogram (ECG) is performed at baseline; days 0, 1, 2, 3 and 5; and then if clinically indicated for ischemic symptoms, or new onset atrial fibrillation or ventricular arrhythmias through Day 30, and the day following the event.

In the follow-up phase following hospital discharge, subjects are contacted by phone on Day 30 and will be assessed for any postoperative MI (Day 6-30), their need for postoperative dialysis, and the occurrence of death. In addition, information regarding re-hospitalization at 30-days along with days and cause of re-hospitalization are obtained. Every effort is made to acquire source documentation for each endpoint. If the subject has died, every effort is made to obtain the circumstances and the cause and date of death. All effort is made via telephone with a representative who has personal knowledge of the subject (e.g., friends, neighbors, primary care physician) to obtain information on the subject's need for postoperative dialysis and their survival status. The study coordinator uses a scripted list of questions to collect information during the telephone contact. If a subject received dialysis, the dates and type of dialysis are recorded from information obtained by contacting the subject or representative and by obtaining documentation from the facility providing dialysis. If the subject has died, every effort should be made to obtain the circumstances and the cause and date of death and information on date of initiation and type of dialysis. Subjects are asked to provide contact information for additional follow-up call required at Day 90. On Day 90, the subject is contacted by phone to determine subject's survival status.

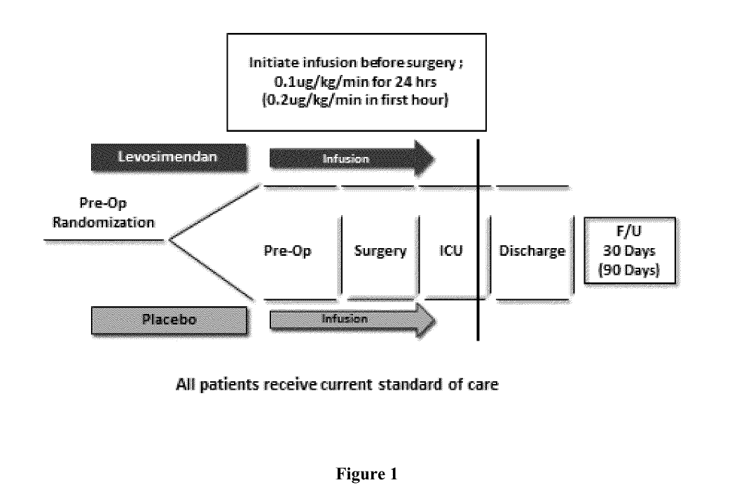

The study schematic is shown in FIG. 1.

### Study Population

Approximately 760 subjects (380 per treatment group) are enrolled in this study. Subjects satisfy the following criteria to be enrolled in the study:
Men or women, 18 years of age or older;
Scheduled to undergo (1) CABG surgery, (2) CABG with aortic valve surgery, (3) CABG with mitral valve surgery, or (4) isolated mitral valve surgery; all patients are on CPB;
Documented LVEF≦25% (for CABG only patients) or LVEF≦35% (CABG with aortic valve, CABG with mitral valve, or isolated mitral valve patients) measured by ventriculogram, echocardiogram (ECHO), nuclear scan, or MRI, within 60 days before surgery;
Signed (by the subjects or their legally acceptable representatives) informed consent document indicating that they understand the purpose of and procedures required for the study and are willing to participate in the study.

Potential subjects who meet any of the following criteria are excluded from participating in the study:
Restrictive or obstructive cardiomyopathy, constrictive pericarditis, restrictive pericarditis, pericardial tamponade, or other conditions in which cardiac output is dependent on venous return;
Pulmonary disease (severe chronic obstructive pulmonary disease [COPD], asthma, or other condition) that, in the opinion of the investigator, represents an independent clinical risk to the cardiac surgery and recovery of the patient;
Evidence of systemic bacterial, systemic fungal, or viral infection within 72 hours before surgery;
Chronic dialysis at baseline or within 30 days of CABG/valve surgery (either hemodialysis, peritoneal dialysis, continuous venovenous hemodialysis);
Estimated glomerular filtration rate (eGFR)<30 mL/kg/min or evidence of worsening renal function before CABG/mitral valve surgery;
Weight≧170 kg;
Patients whose SBP cannot be managed to ensure SBP>90 mmHg at initiation of study drug;
Heart rate≧120 bpm, persistent for at least 10 minutes;
Hemoglobin<80 g/L;
Serum potassium<3.5 mmol/L or >5.5 mmol/L at baseline;
A history of Torsades de Pointes;
Mechanical assist device (IABP, LVAD, ECMO) in previous 30 days or pre-planned use of IABP, LVAD, or ECMO during or following CABG/valve surgery;
Patients with aortal femoral occlusive disease that would prohibit use of IABP;
Liver dysfunction Child Pugh Class B or C;
Patients having severely compromised immune function;
Pregnant, suspected to be pregnant, or breast-feeding;
Received an experimental drug or used an experimental medical device in previous 30 days;
Known allergic reaction or sensitivity to levosimendan or excipients;
Received commercial levosimendan within 30 days before the planned start of study drug;
Employees of the investigator or study center, with direct involvement in the proposed study or other studies under the direction of that investigator or study center, as well as family members of the employees or the investigator; or
Women of childbearing potential agree to remain on an effective method of birth control or remain abstinent throughout the study.

### Randomization and Blinding

Randomization is used to avoid bias in the assignment of subjects to treatment, to increase the likelihood that known and unknown subject attributes (e.g., demographic and baseline characteristics) are evenly balanced across treatment groups, and to enhance the validity of statistical comparisons across treatment groups. Blinded treatment is used to reduce potential bias during treatment, data collection and evaluation of clinical endpoints.

On the day of surgery following acquisition of all required measurements, subjects are randomly assigned to a treatment group using the CDMS after they have met all of the inclusion criteria and none of the exclusion criteria.

Study drug (levosimendan or matching placebo) is initiated as soon as possible prior to surgery, following arterial line incision, and before surgical incision. If insertion of a Swan-Ganz is planned, it is preferable to insert it prior to study drug initiation, or as soon as possible after study drug initiation.

Subjects are assigned to 1 of 2 treatment groups (levosimendan 0.2 μg/kg/min for first hour, followed by 0.1 μg/kg/min for the next 23 hours or matching placebo) in a 1:1 ratio based on a computer-generated randomization schedule prepared before the start of the study by an independent statistical group not otherwise involved in the conduct or analysis of the study.

Randomization is implemented through the CDMS, based on the randomization scheme provided by the unblinded study statistician.

If a potential subject is randomly assigned to treatment but is found to be ineligible before the study drug infusion is started, the investigator does not proceed with study drug administration. The potential subject is considered randomized but not treated, and the sponsor is notified. The investigator documents the reason that the potential subject is no longer a study candidate. The investigator manages the patient's medical condition according to usual clinical practice, and specific study-related procedures (other than those listed below) are not performed. Subjects who are randomly assigned to treatment but not treated are followed through Day 5 for the perioperative MI, and need for mechanical assist (IABP, LVAD, or ECMO); and through Day 30 for postoperative MI (Day 6-10), need for postoperative dialysis, and the occurrence of all-cause mortality.

The study drug container has a 2-part, tear-off label with directions for use and other information on each part. The tear-off section of the label is removed and attached to the subject's drug accountability form when the drug is dispensed. The second part of the label remains affixed to the study drug container and will contain all identifying information except for the dose of the drug contained.

The investigator is not provided with randomization codes. The codes are maintained within the CDMS.

Under normal circumstances, the blind is not broken until all subjects have completed the study (defined as Day 90) and the database for the Day 90 data is locked. Otherwise, the blind is broken only if specific emergency treatment would be dictated by knowing the treatment status of the subject. In such cases, the investigator contacts and informs the sponsor or designee as soon as possible. The date, time, and reason for the unblinding are documented in the appropriate section of the electronic case report form (eCRF) and in the source document.

### Dosage and Administration

Subjects are monitored and their hypotension (mean arterial pressure [MAP]<60 mmHg, SBP<90 mmHg) and signs and symptoms of hypovolemia corrected prior to initiation of study drug. Every attempt is made to start treatment with the study drug as soon as possible prior to surgery, after arterial line insertion, and before skin incision. If insertion of a Swan-Ganz catheter is planned, it is preferable to insert it prior to study drug initiation, or if not already in place, after study drug initiation.

Study drug is administered for 24 hours. The infusion rate of study drug may be decreased, interrupted, or discontinued for safety reasons, according to the discretion of the investigator, as described in the following sections.

The appropriate infusion rate is calculated based on the subject's preoperative body weight.

Levosimendan/placebo infusion for the 24-hour infusion is prepared as follows:

(i) for patients<85 kg, by adding one (1) 5 mL vial of levosimendan/placebo infusion concentrate to one 250 mL infusion bag or bottle of 5% dextrose;

(ii) for patients≧85 kg by adding two (2) 5 mL vials of levosimendan/placebo infusion concentrate to one 500 mL infusion bag or bottle of 5% dextrose.

The concentration of the diluted infusion is about 50 μg/mL in 5% dextrose (12.5 mg/255 mL in a 250 mL bag; 25 mg/510 mL, 2 vials in a 500 mL bag). The i.v. tubing is primed to fill the volume to the i.v. entry site before starting the infusion. The diluted infusion is administered intravenously by a peripheral or central route. No other treatments are administered via the same line, and no bolus of study drug is administered. Study drug is infused as a continuous infusion of 0.2 μg/kg/min over the first 60 minutes. After 1 hour, the flow rate is decreased to a continuous infusion of 0.1 μg/kg/min. This dose of study drug is continued for the next 23 hours. On completion of the 24-hour infusion period, the study drug infusion is switched off abruptly.

During the first 60 minutes, the original dose (0.2 μg/kg/min) may be reduced to 0.1 μg/kg/min and then to 0.05 μg/kg/min as appropriate. During the following 23 hours, the original dose (0.1 μg/kg/min) may be reduced to 0.05 μg/kg/min as appropriate. Patients not tolerating this lowest dose will have their medication permanently discontinued.

Table 3 provides detailed infusion rates for a given patient weight as mL/h for the different periods of administration of a 50 μg/mL preparation of levosimendan/placebo infusion.

TABLE 3
Infusion rates for the different periods of administration
of a 50 μg/mL preparation of levosimendan/placebo infusion

| | | | Reduced |
|---|---|---|---|
| Patients | Infusion rate during | Infusion rate during | infusion rate as |
| weight | first 60 min (mL/h) at | 1 h-24 h (mL/h) at | needed (mL/h) at |
| (kg) | 0.2 μg/kg/min | 0.1 μg/kg/min | 0.05 μg/kg/min |
| 40-44 | 10 | 5 | 2 |
| 45-49 | 11 | 5 | 3 |
| 50-54 | 12 | 6 | 3 |
| 55-59 | 13 | 7 | 3 |
| 60-64 | 14 | 7 | 4 |
| 65-69 | 16 | 8 | 4 |
| 70-74 | 17 | 8 | 4 |
| 75-79 | 18 | 9 | 5 |
| 80-84 | 19 | 10 | 5 |
| 85-89 | 20 | 10 | 5 |
| 90-94 | 22 | 11 | 5 |
| 95-99 | 23 | 11 | 6 |
| 100-104 | 24 | 12 | 6 |
| 105-109 | 25 | 13 | 6 |
| 110-114 | 26 | 13 | 7 |
| 115-119 | 28 | 14 | 7 |
| 120-124 | 29 | 14 | 7 |
| 125-129 | 30 | 15 | 8 |
| 130-134 | 31 | 16 | 8 |
| 135-139 | 32 | 16 | 8 |
| 140-144 | 34 | 17 | 8 |
| 145-149 | 35 | 17 | 9 |
| 150-154 | 36 | 18 | 9 |
| 155-159 | 37 | 19 | 9 |
| 160-164 | 38 | 19 | 10 |
| 165-169 | 40 | 20 | 10 |

If the patient's weight is not indicated in Table 3, the infusion rate is calculated. As an example, for a patient weighing 38 kg:

(i) 0.2 μg/kg/min infusion rate: 0.2×38÷50×60=9 mL/h

(ii) 0.1 μg/kg/min infusion rate: 0.1×38÷50×60=5 mL/h

The infusion rate of study drug is decreased, or interrupted as clinically warranted if the subject has hypotension, tachycardia, or signs or symptoms consistent with hypovolemia (e.g., low SBP, decreasing urine output with rising blood urea nitrogen [BUN] and serum creatinine) not responding to corrective treatment. Time of discontinuation or down titration is recorded. Patients are monitored closely until clinically stable.

The following events occurring at any time during study drug infusion lead to either a dose reduction or temporary discontinuation of the study drug infusion:
Decreases in SBP to ≦80 mmHg (or MAP<55 mmHg) not responding to vasopressors or fluid challenge in 10 minutes;
Heart rate constantly 140 bpm or more for over 10 minutes not related to atrial fibrillation;
The patient that has experienced atrial fibrillation (>140 bpm) lasting for more than 6 h and continues without response to cardioversion and/or 6 h amiodarone treatment;
The physician judges that it is in the best interest of the patient to reduce the dose or discontinue the infusion.

The following patients will their study drug discontinued without dose adjustment:
The patient develops ventricular fibrillation after the end of operation;
A new cardiac operation is warranted due to a suspected surgical graft complication.

Those patients becoming clinically stable following discontinuing study drug may re-initiate study drug as follows.
The infusion can be resumed at half the previous infusion rate (0.05 μg/kg/min). If the event recurs at the dose of 0.05 μg/kg/min then the infusion should be discontinued permanently. The elimination half-life of levosimendan (T_{1.2}=1 hour) should be taken into account when assessing the response to a dose reduction.
Following any dose-reduction the investigator can reinstate a higher dose (maximum 0.1 μg/kg/min) later during the infusion period provided that the basis for the dose reduction has passed and the dose is well tolerated. Even where the study drug infusion has been temporarily discontinued, the study drug infusion must be stopped 24 hours after the initiation of study drug infusion.

### Concomitant Therapy

This study is a comparison of standard care plus levosimendan to standard care plus placebo for the treatment of subjects in the immediate perioperative period. All patients receive standard evidenced based therapies as recommended in local clinical practice guidelines.

The patient's regular per oral concomitant treatments (diuretics, digitalis, angiotensin-converting-enzyme (ACE) inhibitors, nitrates, beta-blockers, and other medications, e.g., antibiotics and analgesics) are administered according to the investigator's clinical judgment.

All subjects receive additional standard of care medications including inotropes, vasopressors, antiarrhythmics, diuretics, nitrates, and nitric oxide as needed. Due to the potentially hypotensive effects of the study drug, the concomitant use of vasodilatory active drugs is used with caution as per the following guidance.

Concomitant administration of nesiritide is not permitted; levosimendan is not initiated within an hour of discontinuing nesiritide therapy.

Hypotension

Provided that hypovolemia as a cause has been ruled out, hypotension, i.e. MAP<60 mmHg, is treated with phenylephrine, ephedrine, norepinephrine, or vasopressin according to investigator's judgment.

Hypertension

Hypertension, i.e., systolic arterial pressure (SAP)>160 mmHg, is treated with bolus doses, or an infusion, of nitroglycerin. Alternatively, sodium nitroprusside is given. However, the concomitant use of nesiritide is not permitted due to its potentially excessive synergistic hypotensive effect with levosimendan.

Low Cardiac Output

To ensure sufficient cardiac output, the volume status, heart rate, blood pressure, and cardiac rhythm of a patient is optimized. The target is to maintain cardiac index>2.2 L/min/m². When a patient's cardiac index falls below 2.0 L/min/m², the first inotropic agent (dobutamine, milrinone, epinephrine, or dopamine) is administered. If this is not sufficient a second inotropic agent is added. In case inotropic drugs cannot maintain cardiac index>2.2 L/min/m², mechanical assist device (IABP/LVAD/ECMO) is considered.

Concomitant treatments from randomization until Day 5 or the end of initial hospitalization are recorded on the eCRF for Concomitant Treatment. No other investigational drug or device should be used with the study drug.

During the 30-day double-blind treatment and post-treatment phases, no use of commercial levosimendan is permitted unless the subject is re-hospitalized for congestive heart failure, where approved for treatment of acute decompensated heart failure.

Concomitant medications are recorded. These include secondary inotropes (dobutamine, milrinone, epinephrine, dopamine), pressors, and anti-arrhythmics. Only medications on this list (prescriptions or over-the-counter medications) continued at the start of the study or started during the study and different from the study drug must be documented. The time of each secondary inotrope's dose (initiation and conclusion) and peak dose is recorded.

### Study Evaluations

All-Cause Mortality

All-cause mortality data (e.g., the cause and date of death) is collected for all subjects up to Day 30 (+5). A follow-up call documents mortality between Day 30 visit and Day 90.

Perioperative Myocardial Infarction (Defined Through Postoperative Day 5)

Serum CK-MB (and CK and troponin) levels are measured at baseline and then following cardiac surgery at time points and for new ischemic events. Similarly, an ECG is obtained at baseline on Days 1, 2, 3, and 5 and for new ischemic events through Day 30. Investigators evaluate and record new MIs identified by new Q waves (>30 ms) and new left bundle branch block on all post-operative ECGs, recognizing that MIs counted as study endpoints will be adjudicated. All post-operative ECG's in patients with peak CK-MB>50 ng/dL, but ≦100 ng/dL are obtained from the site and reviewed by an independent cardiologist blinded to treatment at DCRI. Most peri-operative MIs are classified by algorithm. All MIs (suspected or triggered) are adjudicated using standardized definitions.

Perioperative MIs (through Day 5) are defined as CK-MB>100 ng/mL (or CK-MB>10× ULN) irrespective of ECG changes or CK-MB>50 ng/dL (or CK-MB>5× ULN) with evidence of new Q waves>30 ms in two contiguous leads or new left bundle branch block (LBBB).

Myocardial Infarction (Day 6-30)

Investigators identify any new rise and/or fall of cardiac biomarkers (troponin or CKMB) with at least one value above the URL in subjects with normal baseline values (≦URL); or a rise of >20% if the baseline values are elevated and are stable or falling.

In addition to the elevated biomarkers, one of the following criteria must be present:
Symptoms of ischemia;
New or presumed new significant ST-segment or T wave (ST-T) changes or new LBBB;
New pathological Q waves;
Imaging evidence of new loss of viable myocardium or new regional wall motion abnormality;
Identification of an intracoronary thrombus by angiography or autopsy.

Need for Dialysis (Through Day 30)

Throughout the general term “dialysis” is used to refer to hemodialysis or other forms of dialytic support (e.g., hemodialysis, peritoneal dialysis, continuous venovenous hemodialysis).

Information on postoperative dialysis (e.g., onset and completion date, type of dialytic support) is collected through Day 30.

Use of Mechanical Assist (Through Day 5)

Information on the use of IABP is obtained and recorded for all patients during index hospitalization and procedure. Similarly, the data on use of LVAD (left ventricular, right ventricular or biventricular) is collected. Finally, the use of ECMO in the study is reported.

The endpoint applies to the use of mechanical assist (IABP, LVAD, ECMO) following the start of surgery for poor cardiac function despite inotropic support and optimal fluid balance.

IABP or LVAD use in patients presenting with LCOS following the start of surgery is encouraged. The investigator must identify that the patient has presented with “poor cardiac function despite inotropic support and adequate fluid replacement” in the eCRF.

### Efficacy Criteria

The composite co-primary efficacy endpoints of the trial are the
30-day composite event rate of all-cause death, perioperative MI (through Day 5), need for dialysis (through Day 30), or use of mechanical assist (IABP, LVAD, or ECMO) (through Day 5) tested at two-sided alpha of 0.01; the “quad” co-primary endpoint; and
30-day composite event rate of all-cause death or use of mechanical assist (IABP, LVAD, or ECMO) (through Day 5) tested at two-sided alpha of 0.04; the “dual” co-primary endpoint.

Secondary endpoints of the trial include the:
Duration of ICU/CCU LOS during the index hospitalization;
Incidence of LCOS defined as cardiac index≦2.0 L/min/m² for >30 minutes despite optimal fluid balance and inotropic support (dobutamine, milrinone, epinephrine, dopamine), with the fluid balance and inotropic dose at the investigator's discretion; or
Postoperative use of secondary inotrope (dobutamine, milrinone, epinephrine, dopamine) associated with index surgical procedure.

### Pharmacokinetic Evaluation

Pharmacokinetic assessments are conducted on blood samples collected from patients consenting to participation in the pharmacokinetic substudy. Blood samples (3 ml) for determining the plasma concentrations of levosimendan and its metabolites OR-1855 and OR-1896 are drawn at the conclusion of infusion and at 48 hours following initiation of study drug. Additional samples for pharmacokinetic analysis are collected on Day 5 and Day 10, Day 14 or at hospital discharge should it precede any of these time points.

Acetylation status of patients is genetically determined from a separate blood sample collected at end of drug infusion. In addition, the plasma levels of the levosimendan metabolites is summarized and reported from all samples. The ratio of OR-1896 and OR-1855 is calculated to determine the acetylation status of a subject. This ratio has been shown to reliably predict the acetylation genotype; OR-1896/OR-1855 is >1 in rapid acetylators and <1 in slow acetylators. A substudy of 200 is targeted to provide a sufficient sample size of both acetylator groups.

### Results

Patients who are administered levosimendan during the clinic trial as indicated above show reduced peri- and postoperative morbidity and mortality as compared to patients who receive the placebo.

Patients who are administered levosimendan during the clinic trial as indicated above have a smaller rate of all-cause death at Day 30, as compared to patients who receive the placebo.

Patients who are administered levosimendan during the clinic trial as indicated above have a smaller rate of perioperative MI (through Day 5) as compared to patients who receive the placebo.

Patients who are administered levosimendan during the clinic trial as indicated above have a lesser need for dialysis (through Day 30) as compared to patients who receive the placebo.

Patients who are administered levosimendan during the clinic trial as indicated above have a reduced use of mechanical assist (IABP, LVAD, or ECMO) (through Day 5) as compared to patients who receive the placebo.

### Discussion

This clinical trial determines that levosimendan initiated prior to cardiac surgery and in addition to current standard care, reduces the peri- and postoperative morbidity and mortality in patients at risk for LCOS. This management is particularly important in those cardiac surgery patients whose hearts have little margin for further diminished capacity, the cardiac surgery population at high risk for LCOS characterized by low LVEFs. The cardiac patient population with LVEF<25% (CABG surgery patients) or LVEF≦35% (CABG with aortic valve surgery, CABG with mitral valve surgery, or isolated mitral valve surgery patients) represents those patients at highest risk of reduced cardiac performance (LCOS) through the period of surgical ischemia and its post-surgical impact. Their higher rates of morbidity and mortality, associated with ineffective management of LCOS, provide sufficient power to demonstrate levosimendan's efficacy in the cardiac surgery population.

### REFERENCES

1. Algarni K D et al. “Decreasing prevalence but increasing importance of left ventricular dysfunction and reoperative surgery in prediction of mortality in coronary artery bypass surgery: trends over 18 years.” J Thorac Cardiovasc Surg. 144(2): 340-6 (2011).
2. Antila S et al. “Site dependent bioavailability and metabolism of levosimendan in dogs.” Eur J Pharm Sci. 9: 85-91 (1999).
3. Antila et al. “Clinical pharmacology of levosimendan.” Clin Pharmacokinet 46(7): 535-52 (2007).
4. Eriksson H I et al. “Levosimendan facilitates weaning from cardiopulmonary bypass in patients undergoing coronary artery bypass grafting with impaired left ventricular function.” Ann Thorac Surg. 87(2): 448-54 (2009).
5. Góngora E et al. “Myocardial revascularization with cardiopulmonary bypass.” Cardiac Surgery in the Adult. (Lawrence H. Cohn ed.) (2008).
6. Kivikko M et al. “Pharmacokinetics of levosimendan and its metabolites during and after a 24-hour continuous infusion in patients with severe heart failure.” Int J Clin Pharmacol Ther. 40(10): 465-71 (2002).
7. Kumar G, Iyer P U. “Management of perioperative low cardiac output state without extracorporeal life support: what is feasible?” Ann Pediatr Card. 3: 147-58 (2010).
8. Masse, L. & Antonacci M. “Low cardiac output syndrome: identification and management.” Crit. Care Nurs Clin North Am. 17(4): 375-83 (2005).
9. O'Connor C M et al. “Comparison of coronary artery bypass grafting versus medical therapy on long-term outcome in patients with ischemic cardiomyopathy (a 25-year experience from the Duke Cardiovascular Disease Databank).” Am J Cardiol. 90(2): 101-7 (2002).
10. Shahian D M et al. “The Society of Thoracic Surgeons 2008 cardiac surgery risk models: part 1—coronary artery bypass grafting surgery.” Ann Thorac Surg. 88(1 Suppl): S2-22 (2009).
11. Toller W G & Stranz C. “Levosimendan, a new inotropic and vasodilator agent.” Anesthesiology 104(3): 556-69 (2006).
12. Velazquez E J et al. “Coronary-artery bypass surgery in patients with left ventricular dysfunction.” N Engl J Med. 364(17): 1607-16 (2011).

1. A method of reducing morbidity and mortality in a human patient undergoing cardiac surgery, the method comprising:
(a) a first period of administering to the human patient levosimendan for about 1 hour, wherein the administration of levosimendan during the first period is initiated (i) before skin incision for the cardiac surgery, and (ii) at an infusion dose of about 0.2 μg/kg/min; and
(b) a second period of administering to the human patient levosimendan for about 23 hours, wherein the administration of levosimendan during the second period is initiated at an infusion dose of about 0.1 μg/kg/min.

2. A method of preventing, or reducing the risk of development of, low cardiac output syndrome (LCOS) in a human patient undergoing cardiac surgery, the method comprising:
(a) a first period of administering to the human patient levosimendan for about 1 hour, wherein the administration of levosimendan during the first period is initiated (i) before skin incision for the cardiac surgery, and (ii) at an infusion dose of about 0.2 μg/kg/min; and
(b) a second period of administering to the human patient levosimendan for about 23 hours, wherein the administration of levosimendan during the second period is initiated at an infusion dose of about 0.1 μg/kg/min.

3. A method of reducing the risk of, intensity of, or occurrence of one or more postoperative adverse events in a human patient undergoing cardiac surgery, the method comprising:
(a) a first period of administering to the human patient levosimendan for about 1 hour, wherein the administration of levosimendan during the first period is initiated (i) before skin incision for the cardiac surgery, and (ii) at an infusion dose of about 0.2 μg/kg/min; and
(b) a second period of administering to the human patient levosimendan for about 23 hours, wherein the administration of levosimendan during the second period is initiated at an infusion dose of about 0.1 μg/kg/min.

4. The method of claim 1, wherein the about 0.2 μg/kg/min infusion dose initiated during the first period is decreased to one or more lower infusion doses.

5. The method of claim 4, wherein the about 0.2 μg/kg/min infusion dose administered during the first period is decreased to about 0.1 μg/kg/min.

6. The method of claim 5, wherein the about 0.1 μg/kg/min infusion dose administered during the first period is decreased to about 0.05 μg/kg/min.

7. The method of claim 1, wherein the about 0.1 μg/kg/min infusion dose initiated during the second period is decreased to one or more lower infusion doses.

8. The method of claim 7, wherein the about 0.1 μg/kg/min infusion dose initiated during the second period is decreased to about 0.05 μg/kg/min.

9-15. (canceled)

16. The method of claim 1, wherein the cardiac surgery comprises coronary artery bypass graft (CABG) surgery, CABG with aortic valve surgery, CABG with mitral valve surgery, or isolated mitral valve surgery.

17. The method of claim 1, wherein the cardiac surgery is with a cardiopulmonary bypass.

18-48. (canceled)

49. The method of claim 2, wherein the about 0.2 μg/kg/min infusion dose initiated during the first period is decreased to one or more lower infusion doses.

50. The method of claim 49, wherein the about 0.2 μg/kg/min infusion dose administered during the first period is decreased to about 0.1 μg/kg/min.

51. The method of claim 50, wherein the about 0.1 μg/kg/min infusion dose administered during the first period is decreased to about 0.05 μg/kg/min.

52. The method of claim 2, wherein the about 0.1 μg/kg/min infusion dose initiated during the second period is decreased to one or more lower infusion doses.

53. The method of claim 52, wherein the about 0.1 μg/kg/min infusion dose initiated during the second period is decreased to about 0.05 μg/kg/min.

54. The method of claim 3, wherein the about 0.2 μg/kg/min infusion dose initiated during the first period is decreased to one or more lower infusion doses.

55. The method of claim 54, wherein the about 0.2 μg/kg/min infusion dose administered during the first period is decreased to about 0.1 μg/kg/min.

56. The method of claim 55, wherein the about 0.1 μg/kg/min infusion dose administered during the first period is decreased to about 0.05 μg/kg/min.

57. The method of claim 3, wherein the about 0.1 μg/kg/min infusion dose initiated during the second period is decreased to one or more lower infusion doses.

58. The method of claim 57, wherein the about 0.1 μg/kg/min infusion dose initiated during the second period is decreased to about 0.05 μg/kg/min.
